(19) United States
(12) Patent Application Publication
(10) Pub. No.: US 2024/0302305 A1
(43) Pub. Date: Sep. 12, 2024

(54) DATA ANALYSIS DEVICE, DATA ANALYIS METHOD, PROGRAM, AND RECORDING MEDIUM

(71) Applicant: Sumitomo Electric Industries, Ltd., Osaka-shi, Osaka (JP)

(72) Inventors: Yutaka HOSHINA, Osaka-shi (JP); Kazuya TOKUDA, Osaka-shi (JP); Yoshihiro SAITO, Osaka-shi (JP)

(73) Assignee: Sumitomo Electric Industries, Ltd., Osaka-shi, Osaka (JP)

(21) Appl. No.: 18/279,287

(22) PCT Filed: Apr. 28, 2021
(86) PCT No.: PCT/JP2021/016993

§ 371 Date: Aug. 29, 2023

(51) Int. Cl. G01N 23/2273

(52) U.S. Cl. CPC G01N 23/2273; G01N 2223/6116; G01N 2223/646

(57) ABSTRACT

A data analysis device includes an input unit that receives a measured value of an optoelectronic signal from a photoelectron spectroscopic device measuring the optoelectronic signal generated from a sample by photoelectron spectroscopy and an analysis unit that analyzes the depth profile of the sample by minimizing a sum of square deviations between a theoretical value of the optoelectronic signal and the measured value of the optoelectronic signal using the theoretical value of the optoelectronic signal when the sample is modeled into a multilayer body including a plurality of layers. The analysis unit calculates a relative concentration so as to satisfy a maximum smoothness condition that the relative concentration of the chemical species of the sample smoothly changes in the plurality of layers of the multilayer body in minimizing the sum of square deviations.

### TECHNICAL FIELD

The present disclosure relates to a data analysis device, a data analysis method, a program, and a recording medium.

### BACKGROUND ART

In a semiconductor device represented by a high electron mobility transistor (HEMT), a state of a material in a thickness region of about several nm to several tens nm in a vicinity of an outermost surface of a semiconductor substrate greatly affects a characteristic of the device. For this reason, it is important to evaluate a depth profile in the vicinity of a sample surface in improving the characteristic or analyzing a defect of the semiconductor device.

For example, analysis by scanning transmission electron microscopy (STEM) energy dispersive X-ray analysis (EDX) can evaluate elemental distribution on a sample surface with resolution of an atomic layer level. However, these analyses cannot evaluate a chemical bonding state in the vicinity of the sample surface. Furthermore, because the sample is required to be thinned prior to the analysis, pretreatment of the sample is complicated.

In the case of evaluation by X-ray photoelectron spectroscopy (XPS), a constituent element of the sample and the chemical bonding state of the constituent element can be evaluated without requiring the complicated pretreatment. An information depth that can be evaluated by XPS depends on a measurement condition, and is typically about several nm from the surface. In the case of evaluating the deeper region, etching of the sample surface by ion sputtering is performed. A depth direction profile regarding a composition or a chemical bonding state of the element can be obtained from the spectral information obtained by alternately repeating the ion sputtering and the measurement. However, because there is a possibility that the surface of the sample is damaged by sputtering, there is a possibility that the correct evaluation cannot be performed.

Accordingly, a technique of evaluating the profile in the depth direction of the sample without changing the state of the sample is required. For example, an analysis method in which the maximum entropy method (Maximum Entropy Method, MEM) is applied to data obtained from angle-resolved XPS (ARXPS) acquired without sputtering is proposed. NPL 1 (“Application of Maximum Entropy Method to Semiconductor Engineering”, Yoshiki Yonamoto, Entropy 2013, 15, 1663-1689; doi: 10.3390/e15051663) provides a theoretical description of MEM and an application example to actual XPS data. NPL 2 (“In-depth distribution of elements and chemical bonds in the surface region of calcium-doped diamond-like carbon films”, J. Zemek, J. Houdkova, P. Jiricek, M. Jelinek, K. Jurek, T. Kocourek, and M. Ledinsky, Applied Surface Science 539 148250 (2021)) discloses evaluating a profile in a depth direction by applying MEM to XPS data of a diamond-like carbon (DLC) thin film doped with calcium.

### CITATION LIST

### Non Patent Literatures

NPL 1: “Application of Maximum Entropy Method to Semiconductor Engineering”, Yoshiki Yonamoto, Entropy 2013, 15, 1663-1689; doi: 10.3390/e15051663
NPL 2: “In-depth distribution of elements and chemical bonds in the surface region of calcium-doped diamond-like carbon films”, J. Zemek, J. Houdkova, P. Jiricek, M. Jelinek, K. Jurek, T. Kocourek, and M. Ledinsky, Applied Surface Science 539 148250 (2021)

### SUMMARY OF INVENTION

A data analysis device according to the present disclosure is a data analysis device that analyzes a depth profile of a sample based on a response signal generated from the sample by incidence of a probe, the data analysis device comprising: an input unit that receives a measured value of the response signal from a measurement device measuring the response signal; and an analysis unit that analyzes the depth profile of the sample by minimizing a sum of square deviations between a theoretical value of the response signal and the measured value of the response signal using the theoretical value of the response signal when the sample is modeled into a multilayer body including a plurality of layers, wherein the analysis unit calculates a relative concentration so as to satisfy a maximum smoothness condition that the relative concentration of chemical species of the sample smoothly changes in the plurality of layers of the multilayer body in minimizing the sum of square deviations.

A data analysis method according to the present disclosure comprising: receiving, from a measurement device, a measured value of a response signal generated from a sample by incidence of a probe; and analyzing a depth profile of the sample based on the measured value, wherein the analyzing includes minimizing a sum of square deviations between a theoretical value of the response signal and the measured value of the response signal using the theoretical value of the response signal when the sample is modeled into a multilayer body including a plurality of layers, and the minimizing the sum of square deviations includes calculating a relative concentration so as to satisfy a maximum smoothness condition that the relative concentration of the chemical species of the sample smoothly change in the plurality of layers of the multilayer body.

A program according to the present disclosure causes a computer to execute: receiving, from a measurement device, a measured value of a response signal generated from a sample by incidence of a probe; and analyzing a depth profile of the sample based on the measured value, wherein the analyzing includes minimizing a sum of square deviations between a theoretical value of the response signal and the measured value of the response signal using the theoretical value of the response signal when the sample is modeled into a multilayer body including a plurality of layers, and the minimizing the sum of square deviations includes calculating a relative concentration so as to satisfy a maximum smoothness condition that the relative concentration of chemical species of the sample smoothly change in the plurality of layers of the multilayer body.

A recording medium according to the present disclosure is a recording medium in which a program is recorded, the program causing a computer to execute: receiving, from a measurement device, a measured value of a response signal generated from a sample by incidence of a probe; and analyzing a depth profile of the sample based on the measured value, wherein the analyzing includes minimizing a sum of square deviations between a theoretical value of the response signal and the measured value of the response signal using the theoretical value of the response signal when the sample is modeled into a multilayer body including a plurality of layers, and the minimizing the sum of square deviations includes calculating a relative concentration so as to satisfy a maximum smoothness condition that the relative concentration of chemical species of the sample smoothly change in the plurality of layers of the multilayer body.

### BRIEF DESCRIPTION OF DRAWINGS

FIG. 1 is a schematic diagram illustrating a relationship between a sample and an optoelectronic signal generated in ARXPS.

FIG. 2 is a view illustrating an analysis system including an analyzer according to an embodiment of the present disclosure.

FIG. 3 is a block diagram illustrating a hardware configuration example of a data analysis device of the embodiment of the present disclosure.

FIG. 4 is a view illustrating an example of a functional block of the data analysis device in FIG. 3.

FIG. 5 is a first view illustrating a flow of a maximum smoothness method (MSM) executed by the data analysis device in FIG. 4.

FIG. 6 is a second view illustrating a flow of MSM executed by the data analysis device in FIG. 4.

FIG. 7 is a view illustrating a STEM/EDX analysis result of a sample A.

FIG. 8 is a view illustrating the STEM/EDX analysis result of a sample B.

FIG. 9 is a view illustrating a depth analysis result by Ar sputtering for each of the sample A and the sample B.

FIG. 10 is a view illustrating ARXPS analysis data and XPS analysis data combined with Ar sputtering for each of the sample A and the sample B.

FIG. 11 is a view illustrating an MEM analysis result of a depth profile of the sample A.

FIG. 12 is a view illustrating the MEM analysis result of the depth profile of the sample B.

FIG. 13 is a view illustrating an analysis result of a depth profile of the sample A obtained by applying MSM to ARXPS analysis data.

FIG. 14 is a view illustrating curve fitting of ARXPS analysis data of the sample A.

FIG. 15 is a view illustrating the analysis result of the depth profile of the sample B obtained by applying the MSM to the ARXPS analysis data.

FIG. 16 is a view illustrating the curve fitting of the ARXPS analysis data.

FIG. 17 is a view illustrating another example of a sample to be analyzed by the MSM.

FIG. 18 is a view illustrating a spectrum of an optoelectronic signal generated from the sample in FIG. 17.

FIG. 19 is a view illustrating the analysis result of the depth profile obtained by applying the MSM to XPS analysis data of the sample in FIG. 17.

FIG. 20 is a view illustrating still another example of a sample to be analyzed by the MSM.

FIG. 21 is a view illustrating an analysis result of the depth profile obtained by applying the MSM to the XPS analysis data of the sample in FIG. 20.

### DETAILED DESCRIPTION

### Problem to be Solved by the Present Disclosure

For the purpose of improving a characteristic or an analyzing defect of a semiconductor device product, a sample with an unknown depth profile may be analyzed. However, an initial profile that is almost correct is required in the analysis of the depth profile by the MEM. For this reason, the MEM is not suitable for evaluation of the samples with the unknown depth profile, which is often required in manufacturing industry. The present disclosure provides a technique of evaluating the depth profile of the sample in a non-destructive manner without requiring an accurate initial value (initial profile).

### Advantageous Effect of the Present Disclosure

According to the present disclosure, the depth profile of the sample can be evaluated in the non-destructive manner without requiring the accurate initial value (initial profile).

### Description of Embodiments

First, an embodiment of the present disclosure will be listed and described.
(1) A data analysis device according to an embodiment of the present disclosure is a data analysis device that analyzes a depth profile of a sample based on a response signal generated from the sample by incidence of a probe, the data analysis device includes an input unit that receives a measured value of the response signal from a measurement device measuring the response signal and an analysis unit that analyzes the depth profile of the sample by minimizing a sum of square deviations between a theoretical value of the response signal and the measured value of the response signal using the theoretical value of the response signal when the sample is modeled into a multilayer body including a plurality of layers, in which the analysis unit calculates a relative concentration so as to satisfy a maximum smoothness condition that the relative concentration of chemical species of the sample smoothly changes in the plurality of layers of the multilayer body in minimizing the sum of square deviations.

According to this configuration, the depth profile of the sample can be nondestructively obtained. Furthermore, the relative concentration of the chemical species is calculated so as to satisfy the maximum smoothness condition, a likely analysis result can be obtained. Accordingly, the depth profile of the sample can be obtained without requiring an accurate initial value.
(2) In the configuration of (1), the maximum smoothness condition is a condition that a sum of squares of differences in relative concentrations between adjacent layers is minimized for all chemical species and all layers in the multilayer body.

According to this configuration, the likely analysis result can be obtained for the depth profile of the sample by specifically setting the maximum smoothness condition.
(3) In the configuration of (1) or (2), the analysis unit applies a charge neutral condition related to the chemical species in addition to the maximum smoothness condition in minimizing the sum of square deviations.

According to this configuration, the more likely analysis result can be obtained for the depth profile of the sample. For example, layers that cannot exist in reality can be avoided from appearing in the depth profile.
(4) In the configuration of (3), the analysis unit optimizes a device constant that is a parameter related to the measurement device such that the sum of square deviations between a value obtained by multiplying the theoretical value of the response signal by the device constant and the measured value of the response signal is minimized.

According to this configuration, the device constant is introduced to minimize the sum of square deviations. The theoretical value of the response signal can be brought close to the measured value by optimizing the device constant. Accordingly, the more likely analysis result of the depth profile of the sample can be obtained.
(5) In the configuration of (4), the analysis unit alternately repeats a first arithmetic operation optimizing the relative concentration by fixing the device constant and a second arithmetic operation optimizing the device constant by fixing the relative concentration, and obtains the depth profile from the relative concentration when results of the first arithmetic operation and the second arithmetic operation converge.

When the first arithmetic operation and the second arithmetic operation are simultaneously optimized, the analysis of the depth profile is beyond a scope of a convex secondary planning problem, and thus the initial value becomes a problem that is required. According to this configuration, the first arithmetic operation can be included in a category of the convex secondary planning problem by alternately executing the first arithmetic operation and the second arithmetic operation. On the other hand, the second arithmetic operation is simple four arithmetic operations. Accordingly, the analysis of the depth profile can dispense with an accurate initial value.
(6) In the configuration of any one of (1) to (5), the measurement device is an angular resolution photoelectron spectroscopic device, and the response signal is an optoelectronic signal.

According to this configuration, the depth profile of the sample can be analyzed from measured data obtained by the angle-resolved photoelectron spectroscopy.
(7) A data analysis method according to an embodiment of the present disclosure includes receiving, from a measurement device, a measured value of a response signal generated from a sample by incidence of a probe and analyzing a depth profile of the sample based on the measured value, in which the analyzing includes minimizing a sum of square deviations between a theoretical value of the response signal and the measured value of the response signal using the theoretical value of the response signal when the sample is modeled into a multilayer body including a plurality of layers, and the minimizing the sum of square deviations includes calculating a relative concentration so as to satisfy a maximum smoothness condition that the relative concentration of chemical species of the sample smoothly change in the plurality of layers of the multilayer body.

According to this configuration, the depth profile of the sample can be evaluated in a non-destructive manner without requiring the accurate initial value (initial profile).
(8) In the configuration of (7), the maximum smoothness condition is a condition that a sum of squares of differences in relative concentrations between adjacent layers is minimized for all chemical species and all layers in the multilayer body.

According to this configuration, the likely analysis result can be obtained for the depth profile of the sample by specifically setting the maximum smoothness condition.
(9) In the configuration of (7) or (8) above, the minimizing the sum of square deviations includes applying a charge neutral condition related to the chemical species in addition to the maximum smoothness condition in minimizing the sum of square deviations.

According to this configuration, the more likely analysis result can be obtained for the depth profile of the sample. For example, layers that cannot exist in reality can be avoided from appearing in the depth profile.
(10) In the configuration of (9), the minimizing the sum of square deviations includes optimizing a device constant that is a parameter related to a measurement device measuring the response signal such that the sum of square deviations between a value obtained by multiplying the theoretical value of the response signal by the device constant and the measured value of the response signal is minimized.

According to this configuration, the device constant is introduced to minimize the sum of square deviations. The theoretical value of the response signal can be brought close to the measured value by optimizing the device constant. Accordingly, the more likely analysis result of the depth profile of the sample can be obtained.
(11) In the configuration of (10), the minimizing the sum of square deviations includes repeating alternately a first arithmetic operation of optimizing the relative concentration by fixing the device constant and a second arithmetic operation of optimizing the device constant by fixing the relative concentration until results of the first arithmetic operation and the second arithmetic operation converge and obtaining the depth profile from the relative concentration when the results of the first arithmetic operation and the second arithmetic operation converge.

According to this configuration, the first arithmetic operation can be included in a category of the convex secondary planning problem by alternately executing the first arithmetic operation and the second arithmetic operation. On the other hand, the second arithmetic operation is simple four arithmetic operations. Accordingly, the analysis of the depth profile can dispense with an accurate initial value.
(12) In the configuration of any one of (7) to (10), the measurement device is an angular resolution photoelectron spectroscopic device, and the response signal is an optoelectronic signal.

According to this configuration, the depth profile of the sample can be analyzed from measured data obtained by the angle-resolved photoelectron spectroscopy.
(13) A program according to an embodiment of the present disclosure causes a computer to execute receiving, from a measurement device, a measured value of a response signal generated from a sample by incidence of a probe and analyzing a depth profile of the sample based on the measured value, in which the analyzing includes minimizing a sum of square deviations between a theoretical value of the response signal and the measured value of the response signal using the theoretical value of the response signal when the sample is modeled into a multilayer body including a plurality of layers, and the minimizing the sum of square deviations includes calculating a relative concentration so as to satisfy a maximum smoothness condition that the relative concentration of chemical species of the sample smoothly change in the plurality of layers of the multilayer body.

According to this configuration, the depth profile of the sample can be evaluated using the computer in a non-destructive manner without requiring the accurate initial value (initial profile).
(14) A recording medium according to an embodiment of the present disclosure is a recording medium in which a program is recorded, the program causing a computer to execute receiving, from a measurement device, a measured value of a response signal generated from a sample by incidence of a probe and analyzing a depth profile of the sample based on the measured value, in which the analyzing includes minimizing a sum of square deviations between a theoretical value of the response signal and the measured value of the response signal using the theoretical value of the response signal when the sample is modeled into a multilayer body including a plurality of layers, and the minimizing the sum of square deviations includes calculating a relative concentration so as to satisfy a maximum smoothness condition that the relative concentration of chemical species of the sample smoothly change in the plurality of layers of the multilayer body.

According to this configuration, the depth profile of the sample can be evaluated using the computer in a non-destructive manner without requiring the accurate initial value (initial profile).

### DETAILS OF EMBODIMENT

With reference to the drawings, an embodiment of the present disclosure will be described below. In the drawings, the same or corresponding part is denoted by the same reference numeral, and the description thereof will not be repeated.

The embodiment of the present disclosure is applicable to an analysis method in which some probe such as an X-ray or an electron beam is made incident on the sample and a response signal corresponding to the depth of the sample is detected. Typically, such the analysis method is photoelectron spectroscopy. In the following description, angle-resolved X-ray photoelectron spectroscopy (ARXPS) will be described as an example of the photoelectron spectroscopy.

ARXPS is an analysis technique that substantially changes a detection depth of the sample by changing an inclination angle of the sample with respect to an analyzer. Unlike analysis in a depth direction by ion sputtering, ARXPS can nondestructively analyze a region up to an escape depth of a photoelectron. The information obtained by ARXPS analysis is not the actual depth profile of a chemical specie of the sample, but is an important hint on the depth profile. Accordingly, there is a need for an approach deriving the actual depth profile from the information obtained by the ARXPS analysis. The embodiment of the present disclosure provides the technique for this.

### <1. Theoretical Formula of ARXPS Data>

What is needed first is a theoretical formula linking the depth profile of the sample to the ARXPS analysis data.

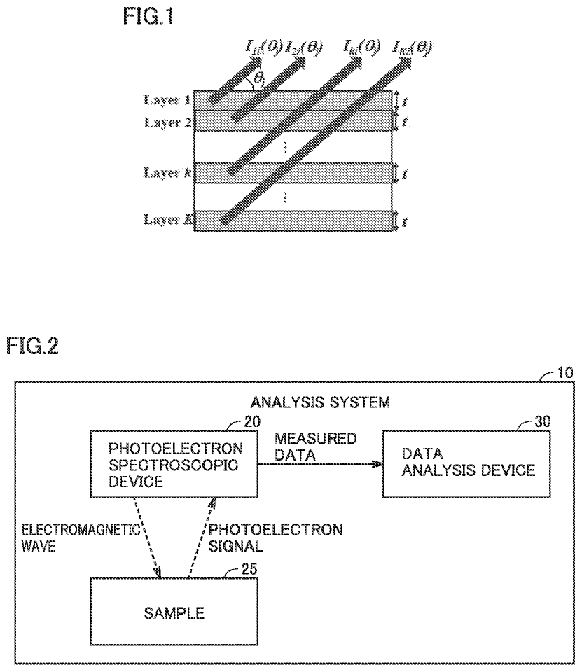

FIG. 1 is a schematic diagram illustrating a relationship between the sample and the optoelectronic signal generated in ARXPS. As illustrated in FIG. 1, the sample is considered to be a stack of K thin layers. A thickness of each layer is t.

It is assumed that the XPS analysis is performed on this sample at a certain extraction angle θⱼ. Extraction angle θⱼ is set to a level J in total. At this point, an optoelectronic signal Iₖᵢ (θⱼ) related to a chemical species i, which is generated in a k-th layer and reaches the sample surface, is expressed by the following equation (1). Here, the number of chemical species i is I in total.

$$\begin{matrix}[ Mathematical formula 1 ] & \\ {I}_{1 i} ( {θ}_{j} ) = {c}_{1 i} {σ}_{i} t & ( 1 )\end{matrix}$$

$${I}_{2 i} ( {θ}_{j} ) = {c}_{2 i} {σ}_{i} texp ( - \frac{t}{{λ}_{1 i} sin {θ}_{j}} )$$

$$⋮$$

$${I}_{ki} ( {θ}_{j} ) = {c}_{ki} {σ}_{i} texp ( - \frac{t}{{λ}_{1 i} sin {θ}_{j}} ) … exp ( - \frac{t}{{λ}_{k - 1 i} sin {θ}_{j}} )$$

$$⋮$$

$${I}_{Ki} ( {θ}_{j} ) = {c}_{Ki} {σ}_{i} texp ( - \frac{t}{{λ}_{1 i} sin {θ}_{j}} ) … exp ( - \frac{t}{{λ}_{K - 1 i} sin {θ}_{j}} )$$

$$that is ,$$

$${I}_{ki} ( {θ}_{j} ) = {c}_{ki} {σ}_{i} t {{\prod }^{k - 1}}_{l = 1} exp ( - \frac{t}{{λ}_{li} sin {θ}_{j}} )$$

In the equation (1), cᵢₖ represents a relative concentration of chemical species i in the k-th layer. A sum of the relative concentrations of chemical species i is 1 (Σᵢcᵢₖ=1). λₗᵢ represents an inelastic mean free step of the photoelectron generated from chemical species i in an l-th layer. σᵢ represents a relative ionization section of the photoelectron of chemical species i with respect to an X-ray. For the signal when k=1, namely, the signal of the outermost surface layer, total power Π is regarded as 1.

It is assumed that a thickness t of the layer is extremely smaller than λₗᵢ (t<<λₗᵢ). Accordingly, in the equation (1), attenuation of the photoelectron in the layer in which the photoelectron is generated is approximated by a linear function (e^{-x} to 1−x).

In the actual ARXPS analysis, the sum of the signals generated from all K layers is observed. Accordingly, a theoretical value d′ᵢⱼ of measured intensity at extraction angle θⱼ for chemical species i is expressed by the following equation (2).

$$\begin{matrix}[ Mathematical formula 2 ] & \\ {d}_{ij}^{′} = {{\sum }^{K}}_{k = 1} {I}_{ki} ( {θ}_{j} ) & ( 2 )\end{matrix}$$

When the equation (1) and the equation (2) are summarized, the relationship between the relative concentration and the XPS theoretical intensity can be expressed by a matrix S and vectors d′,c as in the following equation (3).

$$\begin{matrix}[ Mathematical formula 3 ] & \\ {d}^{′} = Sc & ( 3 )\end{matrix}$$

Vectors d′ and c is expressed by an equation (4) and an equation (5).

$$\begin{matrix}[ Mathematical formula 4 ] & \\ {d}^{′} = {}^{T} ( {d}_{11}^{′} , … , {d}_{1 J}^{′} , {d}_{21}^{′} , … , {d}_{2 J}^{′} , … , {d}_{I 1}^{′} , … , {d}_{IJ}^{′} ) & ( 4 )\end{matrix}$$

$$\begin{matrix}[ Mathematical formula 5 ] & \\ c = {}^{T} ( {c}_{11} , … , {c}_{1 K} , {c}_{21} , … , {c}_{2 K} , … , {c}_{I 1} , … , {c}_{IK} ) & ( 5 )\end{matrix}$$

As illustrated in the equation (4), a vector d′ is a (I×J) row vector in which the ARXPS intensity theoretical values of all chemical species and all angles are arranged in one column. As illustrated in the equation (5), a vector c is a (I×K) row vector in which the relative concentrations of all chemical species and all depths are arranged in one column. Matrix S is a matrix of (I×J) rows and (I×K) columns expressed by the following equation (6). Hereinafter, I×J is referred to as “IJ”, and I×K is referred to as “IK”.

$$\begin{matrix}[ Mathematical formula 6 ] & \\ S = ( \begin{matrix}{S}^{( 1 )} & … & 0 \\ ⋮ & ⋱ & ⋮ \\ 0 & … & {S}^{( I )}\end{matrix} ) {S}^{( i )} = ( \begin{matrix}{r}_{1} {s}_{11}^{( i )} & … & {r}_{1} {s}_{1 K}^{( i )} \\ ⋮ & ⋱ & ⋮ \\ {r}_{J} {s}_{J 1}^{( i )} & … & {r}_{J} {s}_{JK}^{( i )}\end{matrix} ) & ( 6 )\end{matrix}$$

s⁽ⁱ⁾ⱼₖ is expressed according to an equation (7).

$$\begin{matrix}[ Mathematical formula 7 ] & \\ {s}_{jk}^{( i )} = {σ}_{i} t {{\prod }^{k - 1}}_{l = 1} exp ( - \frac{t}{{λ}_{li} sin {θ}_{j}} ) & ( 7 )\end{matrix}$$

A constant rᵢⱼ does not appear in the equation (1) and the equation (2). Constant rᵢⱼ is an important element in the analysis method according to the embodiment of the present disclosure, and will be described in detail later.

In the embodiment, the sum of square deviations between the actual measured data and the theoretical value is used as an index for depth profile evaluation. When the measured data is expressed as a vector d (component is dᵢⱼ) according to the equation (4), the sum of square deviations is expressed according to an equation (8). In the equation (8), a constant ½ is provided for convenience of notation.

$$\begin{matrix}[ Mathematical formula 8 ] & \\ \frac{1}{2} {{\sum }^{I}}_{i = 1} {{\sum }^{J}}_{j = 1} {( {d}_{ij} - {d}_{ij}^{′} )}^{2} = \frac{1}{2} {c}^{T} ( {S}^{T} S ) c - {d}^{T} Sc + \frac{1}{2} {d}^{T} d & ( 8 )\end{matrix}$$

### <2. Problem Regarding Depth Profile Determination>

The depth profile that best reproduces the measured data can be obtained by minimizing the equation (8) with IK relative concentrations cᵢₖ as variables. However, the minimization of the equation (8) has the problem of mathematical extreme instability.

The equation (1) and the equation (2) mean that the intensity of the optoelectronic signal obtained by the ARXPS analysis is the sum of the optoelectronic signals generated from the respective layers of the sample, namely, a weighted average value. The measured data to be fitted with respect to IK relative concentrations cᵢₖ to be obtained is only I depth-direction weighted average values. For this reason, for the minimization problem of the equation (8), a plurality of profiles that are significantly different from each other are candidates for a solution. This means that the solutions may be very different (the solution becomes unstable) even when the measured data fluctuates slightly.

The estimation of IK relative concentrations cᵢₖ from the measured data obtained by the ARXPS analysis corresponds to what is called an inverse problem. According to Jacques Salomon Hadamard, that a generally raised problem is well-posed means that three requirements of (1) existence of a solution, (2) uniqueness of the solution, and (3) continuity or stability of the solution are all satisfied. A missing problem of any one of these requirements corresponds to an inappropriate ill-posed problem.

That the solution to the minimization problem of the equation (8) is not uniquely determined corresponds to “inappropriate problem” in Hadamard's sense. A constraint selecting one solution from an infinite number of solution candidates for the minimization problem of the equation (8) is required in order to obtain the depth profile that reproduces the measured data best.

A reasonable way of thinking (in other words, “common general knowledge”) to some extent exists for the system in FIG. 1. Accordingly, the “common general knowledge” is expressed by a mathematical expression and the addition of the mathematical expression to the equation (8) is minimized, so that the constraint on an infinite number of solution candidates can be imposed.

### <3. Maximum Entropy Method and Problem Thereof>

A maximum entropy method (MEM) requires that “entropy of a system is maximum” as “common general knowledge”. In the case of optimization of IK relative concentrations cᵢₖ, an amount represented by the following equation (9) is considered. The equation (9) expresses the relative entropy of a relative concentration cᵢₖ(0) with respect to relative concentration cᵢₖ (cᵢₖ(0) expresses the initial value of the relative concentration).

$$\begin{matrix}[ Mathematical formula 9 ] & \\ - {{\sum }^{I}}_{i = 1} {{\sum }^{K}}_{k = 1} {c}_{ik} log ( \frac{{c}_{ik}}{{c}_{ik}^{( 0 )}} ) & ( 9 )\end{matrix}$$

The equation (9) can be interpreted as “similarity of relative concentration cᵢₖ with respect to relative concentration cᵢₖ⁽⁰⁾” or “the amount of information obtained by the ARXPS analysis when the estimated state of the relative concentration changes from cᵢₖ⁽⁰⁾ to cᵢₖ”.

The maximization of the relative entropy expressed by the equation (9) results in “looking for an optimal relative concentration cᵢₖ in a range close to initial value cᵢₖ⁽⁰⁾ of the relative concentration”. Accordingly, the determination of initial value cᵢₖ⁽⁰⁾ directly influences the determination of relative concentration cᵢₖ. However, for example, when the ARXPS analysis is performed due to an unknown failure mode generated in the semiconductor device, there is a possibility that initial value cᵢₖ⁽⁰⁾ of the relative concentration cannot be estimated for the sample to be analyzed. In this case, in the MEM, it may be difficult to obtain an accurate evaluation result.

### <4. Maximum Smoothness Method (MSM)>

A new approach to replace the above MEM is referred to herein as a maximum smoothness method (MSM). Application of the MSM to the ARXPS analysis will be specifically described.

The minimization of the amount expressed by the following equation (10) is considered.

$$\begin{matrix}[ Mathematical formula 10 ] & \\ \frac{1}{2} {{\sum }^{I}}_{i = 1} {{\sum }^{K - 1}}_{k = 1} {( {c}_{ik + 1} - {c}_{ik} )}^{2} = \frac{1}{2} {c}^{T} {Q}_{S} c & ( 10 )\end{matrix}$$

At this point, Qₛ is a matrix of IK rows and IK columns expressed by an equation (11).

$$\begin{matrix}[ Mathematical formula 11 ] & \\ {Q}_{S} = ( \begin{matrix}1 & - 1 & 0 & & & & \\ - 1 & 2 & - 1 & … & & 0 & \\ 0 & - 1 & 2 & & & & \\ & ⋮ & & ⋱ & & ⋮ & \\ & & & & 2 & - 1 & 0 \\ & 0 & & … & - 1 & 2 & - 1 \\ & & & & 0 & - 1 & 1\end{matrix} ) & ( 11 )\end{matrix}$$

The equation (10) expresses the sum of squares of the difference in relative concentration between two adjacent layers. The small sum of squares means that a change in relative concentration between layers is smooth. That is, the MSM additionally imposes the constraint that the depth profile is smooth for each chemical species in the equation (8).

The basic idea of the MSM is expressed by the equation (10). In order to obtain a more likely solution, “charge neutral condition” is considered as a further common general knowledge. This is because when only the equation (8) and the equation (10) are a minimization target, the layer that cannot exist in the actual sample may appear in the depth profile. The minimization of the amount expressed by the following equation (12) is considered in order to obtain a likely solution while avoiding such the problem. In the equation (12), eᵢ expresses a constant that restricts an abundance ratio of chemical species i.

$$\begin{matrix}[ Mathematical formula 12 ] & \\ \frac{1}{2} {{\sum }^{K}}_{k = 1} {[ {e}_{1} {c}_{1 k} + {e}_{2} {c}_{2 k} + … + {e}_{I} {c}_{Ik} ]}^{2} = \frac{1}{2} {c}^{T} {Q}_{EN} c & ( 12 )\end{matrix}$$

At this point, Q_{EN} is the matrix of IK rows and IK columns expressed by the following equation (13). Furthermore, E in the equation (13) is a unit matrix of K rows and K columns.

$$\begin{matrix}[ Mathematical formula 13 ] & \\ {Q}_{EN} = ( \begin{matrix}{e}_{1} {e}_{1} E & {e}_{1} {e}_{2} E & & {e}_{1} {e}_{I} E \\ {e}_{2} {e}_{1} E & {e}_{2} {e}_{2} E & … & {e}_{2} {e}_{I} E \\ ⋮ & ⋮ & & ⋮ \\ {e}_{I} {e}_{1} E & {e}_{I} {e}_{2} E & & {e}_{I} {e}_{I} E\end{matrix} ) & ( 13 )\end{matrix}$$

For example, when existence of a chemical species i′ and a chemical species i″ in a ratio of 1:3 in the sample is reasonable from common general knowledge, e_{i′}=3, e_{i″}=−1, and other eᵢ is set to 0 in the equation (12). The signs of e_{i′} and e_{i″} may be opposite to the above signs.

That is, the equation (12) in this case means that in all of the K layers, a penalty is imposed for the concentration ratio of the chemical species i′ and i″ deviating from 1:3.

The sum of the equation (8), the equation (10), and the equation (12) is expressed by an equation (14). In the MSM, the equation (14) is minimized with vector c as a variable. ½d^{T}d in the equation (8) is a constant term that does not depend on vector c, and thus can be ignored in the following discussion.

$$\begin{matrix}[ Mathematical formula 14 ] & \\ \frac{1}{2} {c}^{T} ( {S}^{T} S ) c - {d}^{T} Sc + \frac{λ}{2} {c}^{T} {Q}_{S} c + \frac{{λ}_{EN}}{2} {c}^{T} {Q}_{EN} c & ( 14 )\end{matrix}$$

Parameters λ and λ_{EN} express how strongly we require the profile to be smooth and the charge to be neutral compared to the sum of square deviations, respectively. The smoothness and the degree of charge neutrality of the resulting solution vary depending on parameters λ and λ_{EN}. Because there is no absolute correct answer, the parameter is adjusted so as to obtain a likely solution.

The minimization of the equation (14) corresponds to a quadratic programming problem. In addition, coefficient matrices S^{T}S, Q_{S}, and Q_{EN} of the second-order terms are all semi-positive definite matrices. Thus, the minimization of the equation (14) is not merely the quadratic programming problem but corresponds to a convex quadratic programming problem in which a global optimal solution (a solution that is guaranteed to be best in the entire executable area) is obtained.

The MSM is a method for overcoming the problem that “an initial value needs to be input”, which is a weak point of the MEM. Notably, the MSM eliminates the need for an initial profile altogether by dropping the depth profile decision into the convex quadratic programming problem.

### <5. Method for Conveniently Handling Relative Concentration>

In general, it is difficult to handle absolute value of the signal intensity and absolute concentration of the chemical species in the ARXPS analysis. For this reason, it is common to handle only relative values for both the signal intensity and the concentration of the chemical species. Accordingly, in the calculation of the equation (8), when theoretical value d′ of the ARXPS signal intensity is compared with the experimental data, theoretical value d′ is required to be converted into a relative value.

From the equation (3), theoretical value d′ is linear with respect to relative concentration c. However, in order to convert theoretical value d′ into the relative value, the value obtained by dividing each component of d′ by the total component value of d′ is nonlinear with respect to relative concentration c. Accordingly, the optimization problem of the equation (14) is beyond the scope of the convex quadratic programming problem.

In order to solve this problem, the MSM uses a constant rⱼ as expressed in the equation (6). Constant rⱼ is a separate value for each angle j, and can be regarded as a “device constant” reflecting an unknown element such as absolute sensitivity of the device. That is, constant rⱼ can be regarded as a parameter virtually obtaining theoretical value d′ of the absolute signal intensity from relative concentration c. Naturally, the value of constant rⱼ is unknown, but the value of constant rⱼ can be optimized in parallel with the minimization of the equation (14).

It is assumed that a provisional value of constant rⱼ is obtained at a certain time point of analysis. It is considered that each of constants rⱼ is “updated” by multiplying each of constants rⱼ by a separate constant rⱼ′. The policy of updating constant rⱼ is clear and minimizes the equation (8). This means that the most theoretical value close to the experimental data is derived by optimizing device constant rⱼ.

A submatrix S⁽ⁱ⁾ of matrix S expressed by the equation (6) is expressed by the following equation (15).

$$\begin{matrix}[ Mathematical formula 15 ] & \\ {S}^{′ ( i )} = ( \begin{matrix}{r}_{1}^{′} {r}_{1} {s}_{11}^{( i )} & {r}_{1}^{′} {r}_{1} {s}_{12}^{( i )} & & {r}_{1}^{′} {r}_{1} {s}_{1 K}^{( i )} \\ {r}_{2}^{′} {r}_{2} {s}_{21}^{( i )} & {r}_{2}^{′} {r}_{2} {s}_{22}^{( i )} & … & {r}_{2}^{′} {r}_{2} {s}_{2 K}^{( i )} \\ ⋮ & ⋮ & & ⋮ \\ {r}_{J}^{′} {r}_{J} {s}_{J 1}^{( i )} & {r}_{J}^{′} {r}_{J} {s}_{J 2}^{( i )} & & {r}_{J}^{′} {r}_{J} {s}_{JK}^{( i )}\end{matrix} ) & ( 15 )\end{matrix}$$

Each constant rⱼ contributes only to the angle j component of the experimental and theoretical values. Accordingly, it is assumed that a result obtained by partially differentiating the sum of square deviations of the equation (8) with rⱼ′ is 0. Thus, the update equation expressed by an equation (16) can be obtained.

$$\begin{matrix}[ Mathematical formula 16 ] & \\ {r}_{j}^{′} = \frac{{\sum }_{i = 1}^{I} {d}_{ij} {d}_{ij}^{′}}{{\sum }_{i = 1}^{I} {d}_{ij}^{′ 2}} & ( 16 )\end{matrix}$$

Simultaneous optimization of relative concentration c and constant rⱼ is a problem that an initial value is required beyond the scope of the convex quadratic programming method. However, in the MSM, the optimization of the equation (14) and the update of the equation (16) are alternately performed. The optimization of the equation (14) is the convex quadratic programming problem, and the update of the equation (16) is a simple four arithmetic operation. Accordingly, the construction of the depth profile becomes a problem that does not require the initial value.

### <6. MSM Execution Process>

To summarize the above, the MSM is performed in the steps described below.
(1) The measured data (for example, the ARXPS measured data) is acquired, and the data is converted into a relative value.
(2) σᵢ (relative ionization section) of the equation (1) is set.
(3) The charge neutral condition in the equation (12) is set.
(4) The values of parameters λ and λ_{EN} in the equation (14) are set.
(5) With constant rⱼ=1, rⱼ is updated by the equation (16) to determine the initial value.
(6) The optimization of the equation (14) (the optimization by the convex quadratic programming) and the update of constant rⱼ of the equation (16) are repeated until the result converges.

When σᵢ is multiplied by a constant for all chemical species in step (2), the effect of multiplying σᵢ by the constant is canceled in the first update of rⱼ in step (5). That is, only an accurate relative value needs to be input for σᵢ, and an absolute value that is generally difficult to know details is not required to be input.

### <7. Analyzer and Analysis Method>

In the embodiment of the present disclosure, the computer performs above steps (1) to (6) to analyze the depth profile from the ARXPS analysis data of the sample.

FIG. 2 is a view illustrating an analysis system including an analyzer of the embodiment of the present disclosure. An analysis system 10 includes a photoelectron spectroscopic device 20 and a data analysis device 30.

Photoelectron spectroscopic device 20 irradiates a sample 25 with an electromagnetic wave as a probe, and measures the intensity of an optoelectronic signal (response signal) generated from sample 25. In one embodiment, photoelectron spectroscopic device 20 is a device that performs angle-resolved photoelectron spectroscopy (for example, ARXPS). Sample 25 is a solid sample such as a semiconductor device.

Data analysis device 30 is implemented by hardware according to a general-purpose computing architecture. Data analysis device 30 acquires the measured data from photoelectron spectroscopic device 20. Data analysis device 30 applies the MSM to the measured data to analyze the depth profile of sample 25.

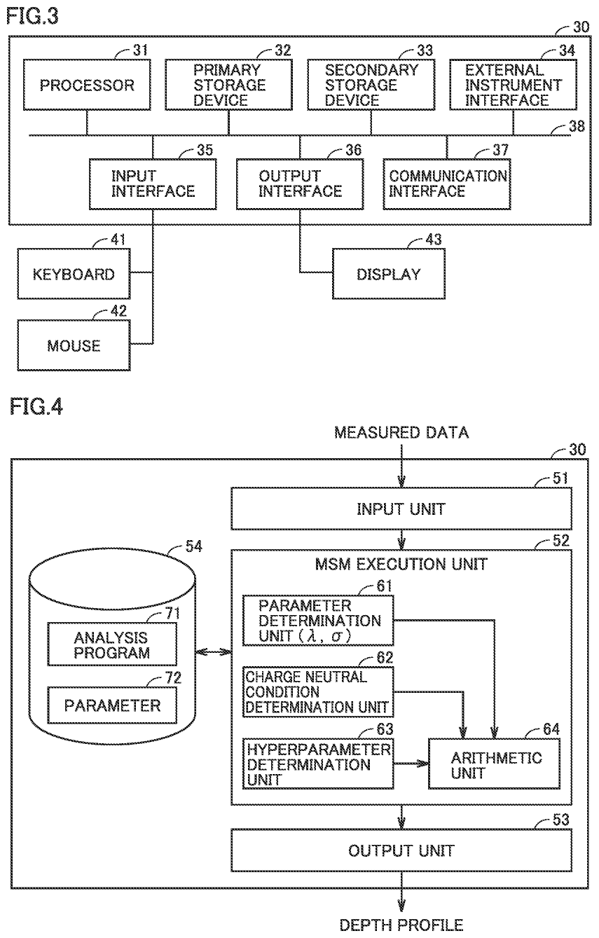

FIG. 3 is a block diagram illustrating a hardware configuration example of the data analysis device of the embodiment of the present disclosure. Data analysis device 30 includes a processor 31, a primary storage device 32, a secondary storage device 33, an external instrument interface 34, an input interface 35, an output interface 36, a communication interface 37, and a bus 38. Elements such as processor 31 and primary storage device 32 exchange data, signals, and the like through bus 38.

Processor 31 processes the program and data stored in primary storage device 32.

Primary storage device 32 stores the program executed by processor 31 and the data referred to. In one aspect, a dynamic random access memory (DRAM) may be used as primary storage device 32.

Secondary storage device 33 stores programs, data, and the like in a nonvolatile manner. In one aspect, a non-volatile storage device such as a hard disk drive (HDD) and a solid state drive (SSD) may be used as secondary storage device 33. Accordingly, secondary storage device 33 corresponds to a computer-readable recording medium in which the program executed by the computer is recorded.

External instrument interface 34 is used when an auxiliary device is connected to data analysis device 30. For example, external instrument interface 34 is a universal serial bus (USB) interface.

Input interface 35 is used to connect input devices such as a keyboard 41 and a mouse 42. Input interface 35 receives a user operation and a user input through these input devices.

Output interface 36 is used to connect an output device such as a display 43.

Communication interface 37 is used to communicate data analysis device 30 to the external instrument. For example, communication interface 37 is used for communication of data analysis device 30 through a network. The communication with the external device may be either wireless communication or wired communication.

Data analysis device 30 may optionally have an optical drive. The optical drive reads the computer-readable program from a recording medium (for example, an optical recording medium such as a digital versatile disc (DVD)) that non-transiently stores the program. The program read from the recording medium may be installed in secondary storage device 33 or the like. In addition, various programs executed by data analysis device 30 may be downloaded from a server device or the like on a network, and installed in data analysis device 30.

FIG. 4 is a view illustrating an example of a functional block of data analysis device 30 in FIG. 3. In one aspect, each block in FIG. 4 is implemented by the computer that executes the program according to the embodiment of the present disclosure.

As illustrated in FIG. 4, data analysis device 30 includes an input unit 51, an MSM execution unit 52, an output unit 53, and a storage unit 54.

Input unit 51 receives the measured data output from photoelectron spectroscopic device 20 (see FIG. 2). Input unit 51 further receives various types of information (for example, information regarding a type of a material and a chemical species) required to analyze the depth profile of sample 25.

Storage unit 54 stores an analysis program 71 for the depth profile of sample 25, a parameter 72 required to execute the MSM, and the like. Furthermore, storage unit 54 may store the measured data input to data analysis device 30.

MSM execution unit 52 is an analysis unit that executes the above-described MSM to obtain the depth profile of sample 25. MSM execution unit 52 includes a parameter determination unit 61, a charge neutral condition determination unit 62, a hyperparameter determination unit 63, and an arithmetic unit 64.

Parameter determination unit 61 determines the values of an inelastic mean free process λ and a relative ionization section σ included in the equation (6) and the equation (7). Charge neutral condition determination unit 62 determines the charge neutral condition for the sample to be analyzed. Specifically, charge neutral condition determination unit 62 determines the value of the parameter e included in the equation (13). Hyperparameter determination unit 63 determines the values of parameters λ and λ_{EN} included in the equation (14).

Arithmetic unit 64 receives the parameter values determined by parameter determination unit 61, charge neutral condition determination unit 62, and hyperparameter determination unit 63, and calculates relative concentration c for all the chemical species and all the layers. Thus, the depth profiles of all chemical species are determined.

Output unit 53 outputs the depth profile of sample 25 obtained by MSM execution unit 52 as the analysis result. For example, the analysis result is displayed on display 43 (see FIG. 3).

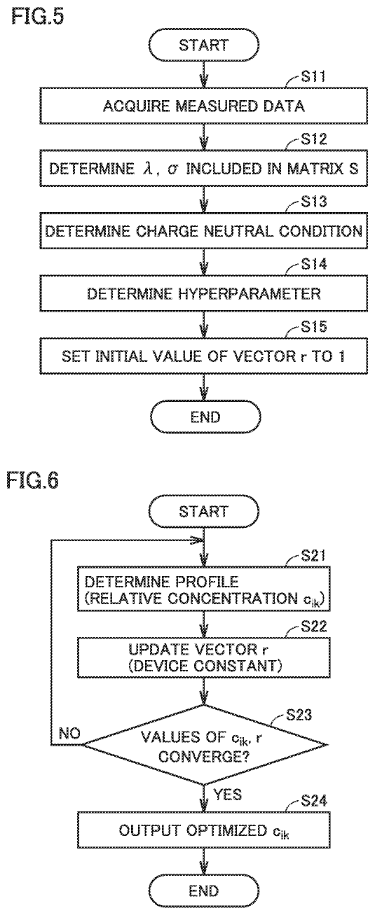

FIG. 5 is a first view illustrating a flow of the MSM executed by data analysis device 30 in FIG. 4. FIG. 6 is a second view illustrating the flow of the MSM executed by data analysis device 30 in FIG. 4.

The flow in FIG. 5 is a flow related to various settings performing the arithmetic operation of the relative concentration. In step S11, input unit 51 acquires the measured data from photoelectron spectroscopic device 20. MSM execution unit 52 executes pieces of processing in steps S12 to S15 described below. For convenience of description, in FIG. 5, the pieces of processing in steps S12 to S15 are described to be executed in order. However, steps S12 to S15 may be executed in an arbitrary order. Furthermore, the pieces of processing in a plurality of steps may be simultaneously executed.

In step S12, parameter determination unit 61 determines the values of inelastic mean free process λ and relative ionization section σ included in matrix S (see the equation (6) and the equation (7)).

The values of inelastic mean free step 2 and relative ionization section σ depend on the material constituting sample 25. For example, storage unit 54 may store a database that defines the values of inelastic mean free process 2 and relative ionization section σ for each type of material. Parameter determination unit 61 can acquire the values of inelastic mean free process λ and relative ionization section σ by referring to the database.

As described above, the value of inelastic mean free process λ is strictly different from each layer. However, inelastic mean free step λ can be set to a constant value in all layers from the viewpoint of saving calculation cost and time by MSM execution unit 52. On the other hand, A can also be a separate value for each layer in order to obtain higher estimation accuracy. For example, a method such that a plurality of kinds of substances of which the value of A is known are previously selected, the relative concentration of each layer is approximated by a linear combination of the plurality of kinds of substances in each time the relative concentration c is optimized by the equation (14), and calculation is advanced while λ in the layer is expressed by the linear combination of the same ratio can be adopted. The value of relative ionization section σ may be a relative value.

In step S13, charge neutral condition determination unit 62 determines the charge neutral condition. Specifically, charge neutral condition determination unit 62 determines a combination of optoelectronic signals limiting the degree of freedom of the composition (for example, SiN or GaN) of the sample. For example, charge neutral condition determination unit 62 can determine the combination of the optoelectronic signals based on composition information input to input unit 51. When determining the combination of the optoelectronic signals, charge neutral condition determination unit 62 determines the values and signs of parameters eᵢ to eₗ included in the equation (13).

In step S14, hyperparameter determination unit 63 determines the values of parameters λ and WEN included in the equation (14). As an example, hyperparameter determination unit 63 determines the value of parameter λ to an arbitrary value and determines the value of parameter λ_{EN} to an arbitrary value. The values of parameters λ and λ_{EN} are not changed until the analysis of the depth profile is finished.

In step S15, arithmetic unit 64 determines the initial value of vector r (device constant). For example, rⱼ is set to 1. The initial value of vector r can be randomly determined. This is because when the first calculation loop is executed according to the flow in FIG. 6, the influence of the magnitude of the value of rⱼ is reset by the processing in step S22. For example, when the initial value of rⱼ is set to 100 instead of 1, each rⱼ is multiplied by a ratio of 1/100 of the original value in the processing in step S22.

The flow in FIG. 6 is a flow related to the arithmetic operation of the relative concentration. In step S21, arithmetic unit 64 determines the profile (relative concentration cᵢₖ) while fixing the device constant. The processing in step S21 is the minimization using vector c as a variable in the equation (14). The processing of step S21 corresponds to the obtainment of the solution of the convex secondary planning problem. Accordingly, various algorithms known as algorithms obtaining the solution of the convex quadratic programming problem can be applied to the processing in step S21.

In step S22, arithmetic unit 64 updates vector r (device constant) while fixing the profile (relative concentration cᵢₖ). Specifically, arithmetic unit 64 updates constant rⱼ′ according to the equation (16). The processing in step S22 corresponds to the optimization of vector r (device constant).

In step S23, arithmetic unit 64 determines whether the values of relative concentration cᵢₖ and vector r converge. When the absolute value of the difference between the “the value of r obtained in the previous calculation” and the “the value of r obtained in the current calculation” is less than or equal to the predetermined value, arithmetic unit 64 determines that the value of vector r converges. In this case, the processing proceeds to step S24. On the other hand, when the value of vector r does not converge, the processing returns to step S21. Accordingly, the arithmetic operation (first arithmetic operation) optimizing relative concentration cᵢₖ and the arithmetic operation (second arithmetic operation) optimizing device constant r are alternately repeated until the value of vector r converges.

In step S24, arithmetic unit 64 outputs optimized relative concentration cᵢₖ. Thus, the depth profile is obtained.

### <8. Example of Depth Profile Analysis>

### Example 1: SiON Thin Film Sample Formed on Si Wafer

An example of the depth profile analysis for a SiON thin film formed on a Si wafer is illustrated. The depth profile analysis will be described below for two types of samples having different thicknesses of the SiON thin film.

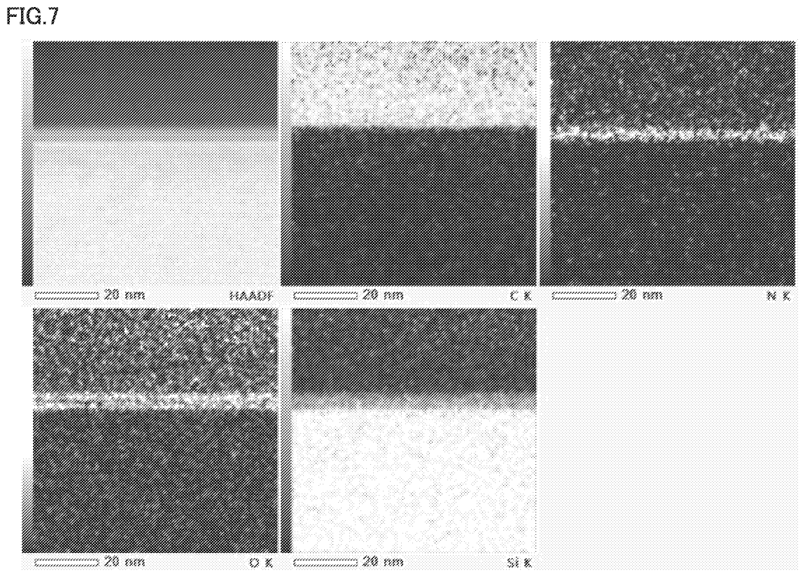

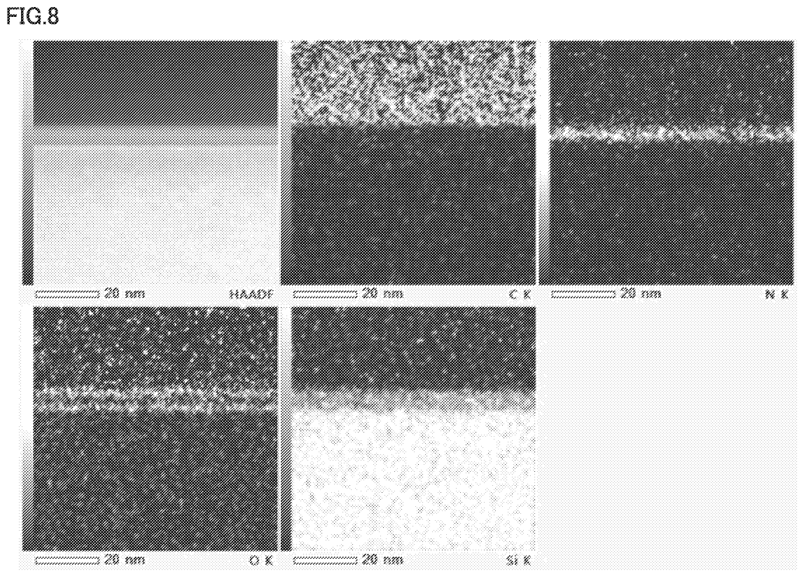

The results of STEM/EDX analysis on the sample are illustrated for comparison with the ARXPS analysis. FIG. 7 is a view illustrating the STEM/EDX analysis result of a sample A. FIG. 8 is a view illustrating the STEM/EDX analysis result of a sample B. For example, FIG. 8 illustrates that in sample B, the thickness of the SiON film is about 7 nm, and a large amount of oxygen (O) element exists on both a surface side and a wafer side of the SiON film.

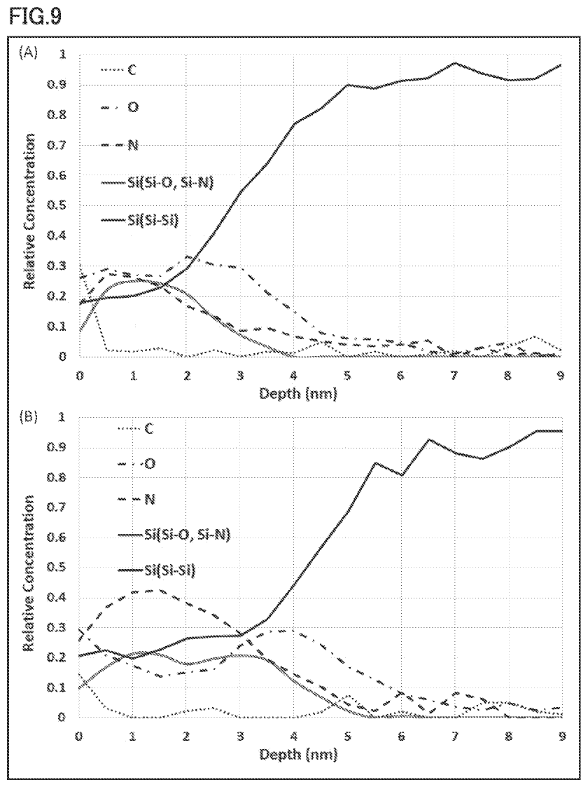

The depth analysis using ion sputtering is a commonly-performed method. FIG. 9 is a view illustrating a depth analysis result by Ar sputtering for each of sample A and sample B. FIG. 9(A) illustrates the depth analysis result of sample A by the Ar sputtering. FIG. 9(B) illustrates the depth analysis result of sample B by the Ar sputtering.

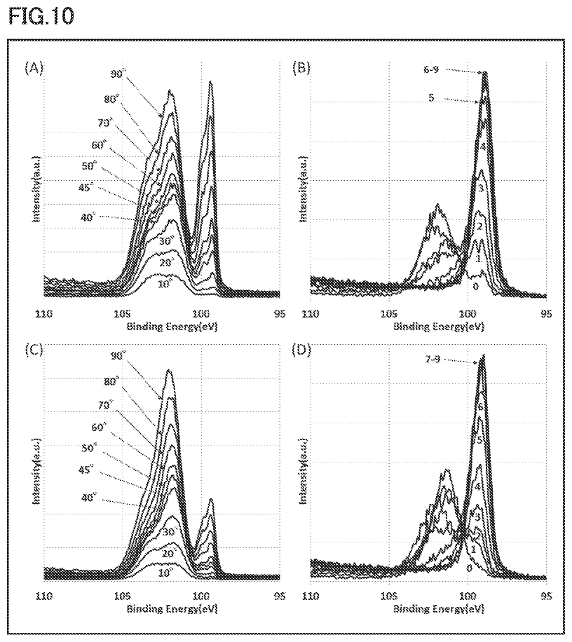

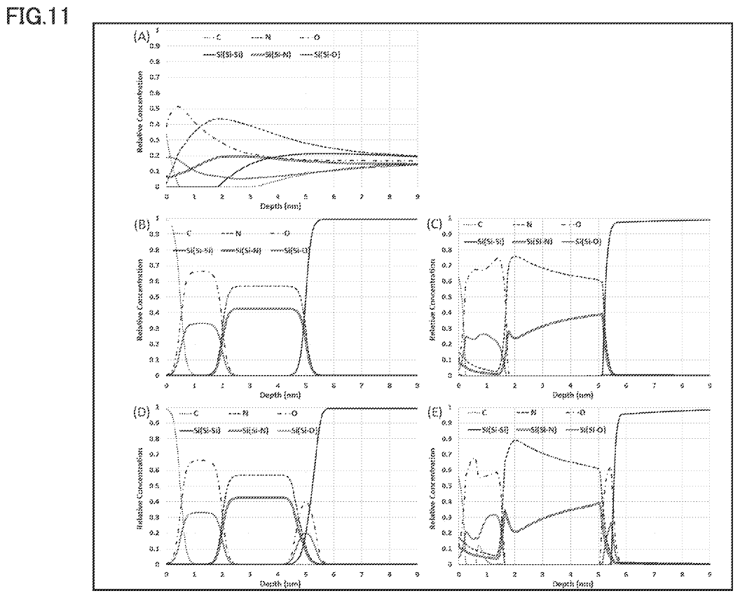

FIG. 10 is a view illustrating the ARXPS analysis data and the XPS analysis data combined with the Ar sputtering for each of sample A and sample B. For the ARXPS analysis, “QuanteraSXM” manufactured by ULVAC PHI was used, and Al Kα rays (1487 eV) were used as an X-ray source. The photoelectron spectra in FIGS. 10(A) to 10(D) are all photoelectron spectra of Si2p.

FIGS. 10(A) and 10(B) illustrate the ARXPS analysis data and the XPS analysis data in combination with the Ar sputtering of sample A. FIGS. 9(C) and 9(D) illustrate the ARXPS analysis data and the XPS analysis data in combination with the Ar sputtering of sample B. The ARXPS analysis was performed using “QuanteraSXM” manufactured by ULVAC PHI, Inc. using Al Kα rays (1487 eV) as the X-ray source at an extraction angle of 10° to 90° in increments of 10° and at a total of 10 levels of 45°.

The problems caused by the combined use of ion sputtering will be specifically described. As illustrated in FIGS. 10(A) and 10(C), in the data of the ARXPS analysis, three peaks of Si—Si (99 eV), Si—N (102 eV), and Si—O (104 eV) clearly appear. On the other hand, as illustrated in FIGS. 10(B) and 10(D), in the XPS analysis data combined with the Ar sputtering, the peak of Si—N and the peak of Si—O overlap each other. For this reason, it is difficult to distinguish between the two peaks. This is because the state of elements in the SiON film was changed by the sputtering. Accordingly, it can be seen that the depth analysis by the Ar sputtering is not suitable for the accurately evaluation of the above system.

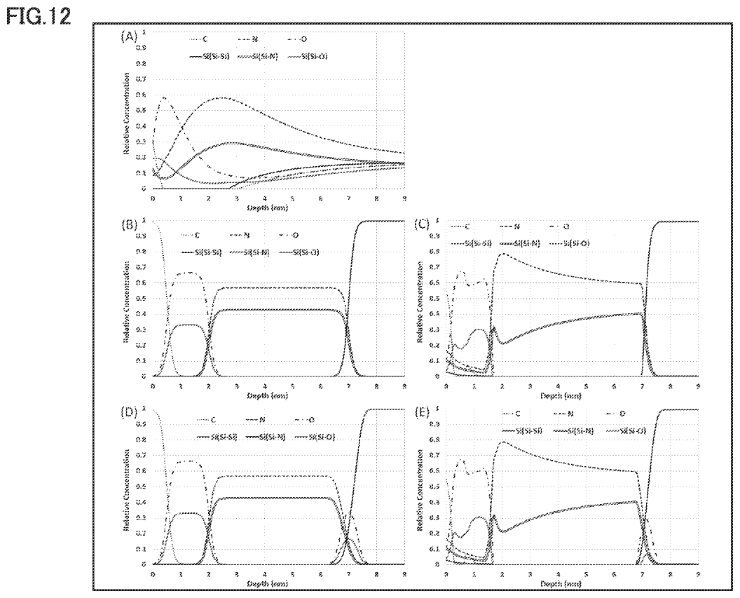

FIGS. 11 and 12 illustrate the analysis results of the depth profile obtained by applying the MEM to the ARXPS analysis data of the sample. FIG. 11 is a view illustrating the MEM analysis result of the depth profile of sample A. FIG. 12 is a view illustrating the MEM analysis result of the depth profile of sample B.

FIGS. 11(A) and 12(A) illustrate the depth profiles obtained assuming uniform (all ⅙) initial distribution of the chemical species (carbon (C), nitrogen (N), oxygen (O), silicon (Si)). For silicon, it was assumed that (Si—Si), (Si—O), and (Si—N) were uniformly distributed. In the profile of FIG. 11(A), SiN, Si, and the like are not clearly divided into layers and are uniform as a whole, resulting in almost the same initial distribution.

FIGS. 11(B) and 12(B) illustrate a first example of the initial distribution of (C/SiO/SiN/Si). FIGS. 11(C) and 12(C) illustrate the depth profile obtained by the first example of the initial distribution of (C/SiO/SiN/Si).

FIGS. 11(D) and 12(D) illustrate a second example of the initial distribution of (C/SiO/SiN/Si). FIGS. 11(E) and 12(E) illustrate the depth profile obtained by the second example of the initial distribution of (C/SiO/SiN/Si).

From FIGS. 11 and 12, it can be seen that in the MEM, the initial distribution close to the correct answer is required to obtain the accurate depth profile. For example, as illustrated in FIGS. 11(D) and 11(E), when the initial distribution that a thin SiO film exists at an interface between the SiN film and the Si wafer is defined, the SiO film appears at the interface between the SiN film and the Si wafer also in the analysis result. However, as illustrated in FIGS. 11(B) and 11(C), when the SiO film at the interface between the SiN film and the Si wafer is not defined as the initial distribution, the SiO film cannot be reproduced in the analysis result. The same applies to the analysis result in FIG. 12.

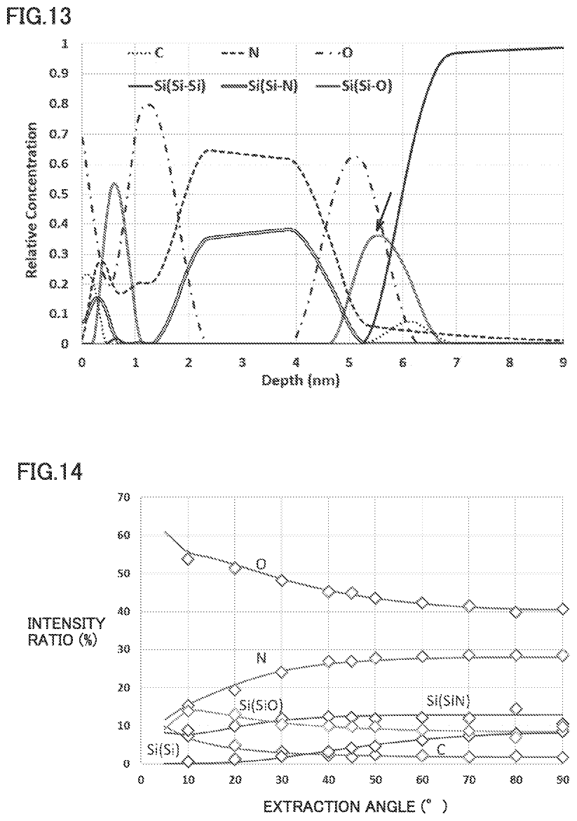

FIGS. 13 to 16 illustrate the analysis results of the depth profile obtained by applying the MSM to the ARXPS analysis data of the sample. In the application of the MSM, the optoelectronic signal of silicon (Si) is separated into the optoelectronic signal of each of Si—Si, Si—O, and Si—N by curve fitting of the ARXPS analysis data. Furthermore, the charge neutral condition is set under the assumption of Si₃N₄ for the charge neutral condition of the equation (12). Furthermore, for hyperparameters λ and λ_{EN}, λ=1 and λ_{EN}=1.

FIG. 13 is a view illustrating the analysis result of the depth profile of sample A obtained by applying the MSM to the ARXPS analysis data. FIG. 14 is a view illustrating the curve fitting of the ARXPS analysis data of sample A.

As indicated by the arrows in FIG. 13, the analysis result of the MSM illustrates that the SiO film exists at the interface between the SiN layer and the Si wafer. This result is consistent with the STEM/EDX analysis result. Furthermore, the thickness of the SiN layer also agrees well with the STEM/EDX analysis results.

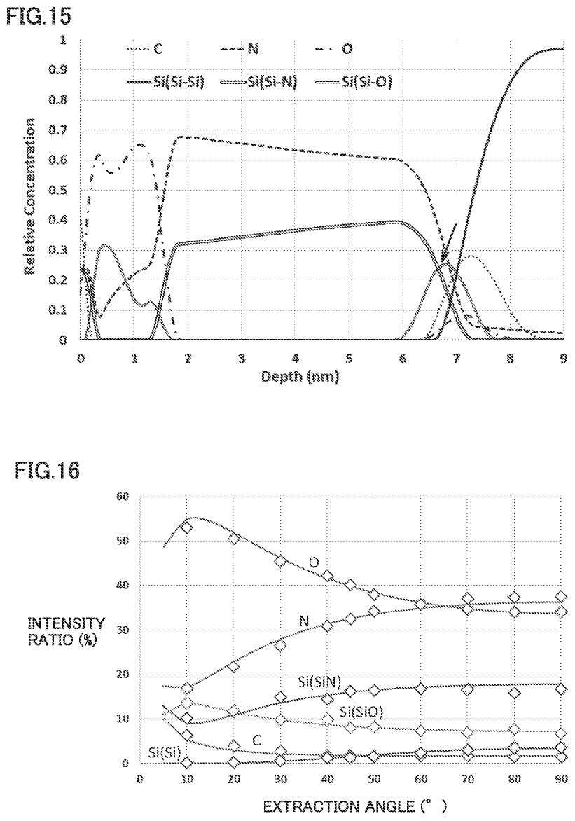

FIG. 15 is a view illustrating the analysis result of the depth profile of sample B obtained by applying the MSM to the ARXPS analysis data. FIG. 16 is a view illustrating the curve fitting of the ARXPS analysis data. As indicated by the arrows in FIG. 15, also in the case of sample B, the analysis result of the MSM indicates that the SiO film exists at the interface between the SiN layer and the Si wafer. Furthermore, the thickness of the SiN layer also agrees well with the STEM/EDX analysis results.

### Example 2: Au/SiN/InP Sample

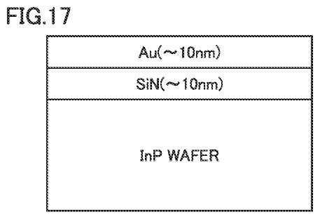

FIG. 17 is a view illustrating another example of the sample to be analyzed by the MSM. As illustrated in FIG. 17, in the second example, the sample is an indium phosphide (InP) substrate with the SiN film formed on the surface. BL16XU of Spring 8 was used for the XPS measurement. The X-rays were monochromatized to 8000 eV using a Si 111 crystal bispectral spectrometer and a Si 444 channel cut spectrometer. R4000 manufactured by Scienta Omicron was used as an optoelectronic analyzer.

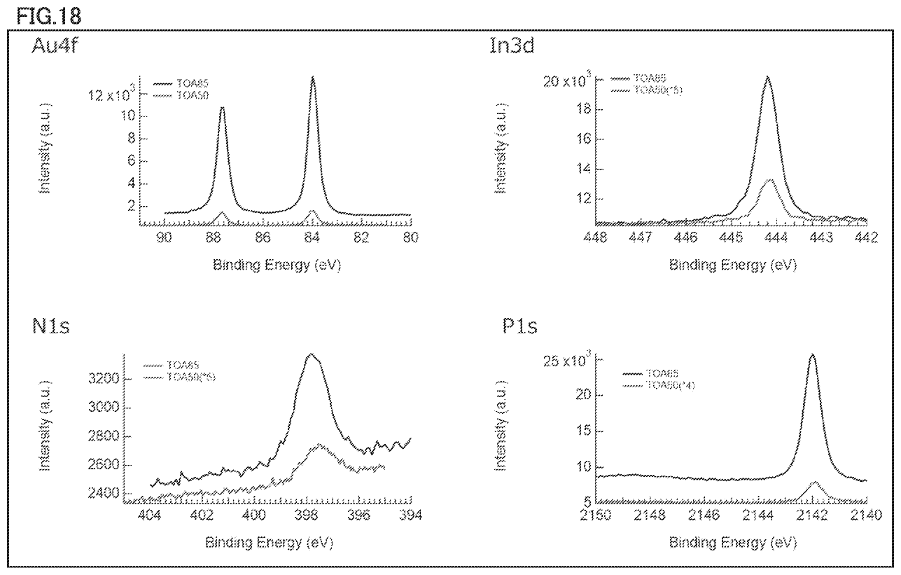

FIG. 18 is a view illustrating a spectrum of the optoelectronic signal generated from the sample in FIG. 17. FIG. 18 illustrates spectra of the optoelectronic signals of Au4f, N1f, In3d, P1s. It can be seen from FIG. 18 that information from a deep position of the sample is obtained. When the composition of the sample surface is SiN, it is not easy to neutralize charging of the sample during the measurement. For this reason, the sample in which the surface was coated with a gold (Au) film was used for the measurement of the optoelectronic signal. The SiN film has the thickness of about 10 nm at maximum, and the Au film has the thickness of about 10 nm at maximum. The extraction angles of the optoelectronic signals are 50° and 85°.

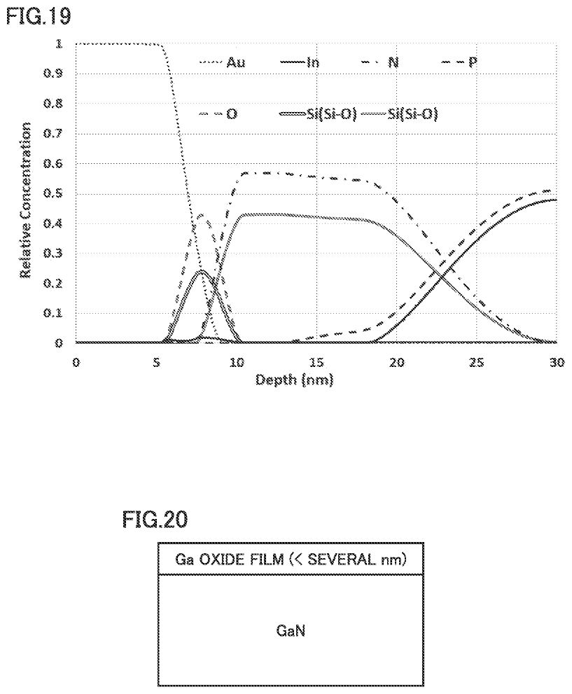

FIG. 19 is a view illustrating the analysis result of the depth profile obtained by applying the MSM to the XPS analysis data of the sample in FIG. 17. The entire structure of Au/SiN/InP can be derived only from information of the two angles. In order to reflect the prior information that “the composition of the sample surface is Au”, at an angle of 10°, dummy data that gold (Au) is 100% is added to the analysis of the depth profile. In the obtained analysis result, the information that the SiO film exists on the surface of the SiN film is correctly expressed.

### Example 3: GaN Wafer Surface with Different Processing Condition

FIG. 20 is a view illustrating still another example of the sample to be analyzed by the MSM. As illustrated in FIG. 20, in a third example, the sample is a gallium nitride (GaN) substrate having a gallium (Ga) oxide film formed on the surface of the gallium nitride substrate. Furthermore, a plurality of samples having different states of the surface of the gallium nitride substrate were prepared by varying the processing condition of the gallium nitride substrate. Specifically, the thickness of the gallium oxide film is different. The thickness of the gallium oxide film is estimated to be less than or equal to several nm.

For the XPS measurement, BL17 of the synchrotron radiation facility Kyushu Synchrotron Light Research Center was used. The X-ray was monochromatized to a photon energy of 600 eV using a variable-angle diffraction grating spectrometer. The number of notches of the diffraction grating was 1000/mm. R3000 manufactured by Scienta Omicron was used as an optoelectronic analyzer. The extraction angles of the optoelectronic signals are 30°, 45° and 85°.

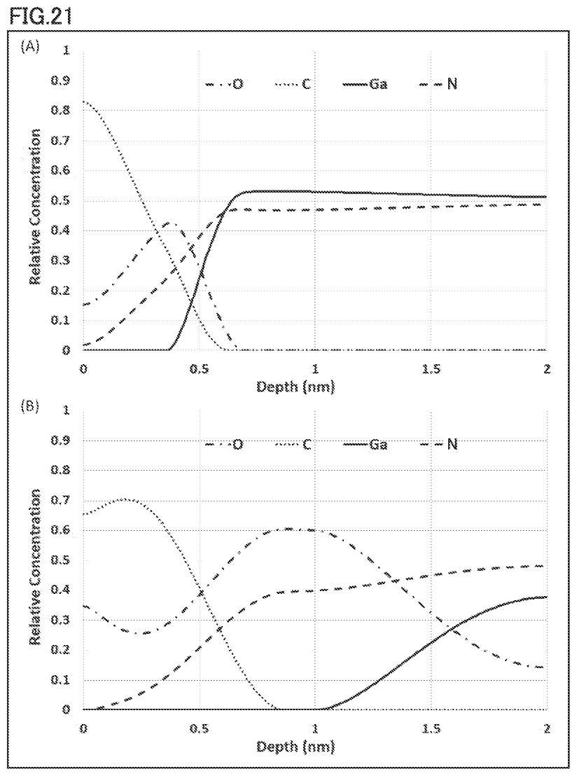

FIG. 21 is a view illustrating an analysis result of the depth profile obtained by applying the MSM to the XPS analysis data of the sample in FIG. 20. FIGS. 21(A) and 21(B) illustrate the analysis results of two types of samples processed under different conditions. As described above, the two kinds of samples are different in terms of the thickness of the Ga oxide film.

As illustrated in FIG. 21, the entire structure of the sample, namely, the contamination of the substrate surface, the Ga oxide film, and the GaN bulk are derived only by the information of the three extraction angles. Furthermore, FIG. 21(A) illustrates that the thin Ga oxide film exists in the sample. On the other hand, FIG. 21 (B) illustrate that the thick Ga oxide film exists in the sample. From FIGS. 21(A) and 21(B), it can be seen that the difference in the thickness of the Ga oxide film due to the difference in the processing condition is correctly expressed.

As illustrated by the above example, the MSM can obtain the likely analysis result for the depth profile of the sample while the need for complicated pretreatment of the sample such as the STEM/EDX analysis is eliminated. Accordingly, the depth profile of the sample can be analyzed more easily than the conventional analysis method.

Furthermore, the MSM can obtain the likely analysis result even though the accurate initial value is unnecessary. Thus, the MSM can be applied to various situations requiring the analysis of the depth profile of the sample, for example, device development, defect analysis, and the like.

In the present specification, the example of the XPS is mainly illustrated as the application example of the MSM. However, the application of the MSM is not limited to the XPS, but is universally applicable to the analysis capable of changing a penetration depth of the probe into the sample, for example, EDX analysis or X-ray fluorescence analysis (XRF).

Although the embodiment of the present invention has been described, it should be considered that the disclosed embodiment is an example in all respects and not restrictive. The scope of the present invention is indicated by the claims, and it is intended that all modifications within the meaning and scope of the claims are included in the present invention.

### REFERENCE SIGNS LIST

10: analysis system, 20: photoelectron spectroscopic device, 25: sample, 30: data analysis device, 31: processor, 32: primary storage device, 33: secondary storage device, 34: external instrument interface, 35: input interface, 36: output interface, 37: communication interface, 38: bus, 41: keyboard, 42: mouse, 43: display, 51: input unit, 52: MSM execution unit, 54: storage, 53: output unit, 61: parameter determination unit, 62: charge neutral condition determination unit, 63: hyperparameter determination unit, 64: arithmetic unit, 71: analysis program, 72: parameter, S11 to S15, S21 to S24: step

1. A data analysis device that analyzes a depth profile of a sample based on a response signal generated from the sample by incidence of a probe, the data analysis device comprising:
an input circuitry that receives a measured value of the response signal from a measurement device measuring the response signal; and
an analysis circuitry that analyzes the depth profile of the sample by minimizing a sum of square deviations between a theoretical value of the response signal and the measured value of the response signal using the theoretical value of the response signal when the sample is modeled into a multilayer body including a plurality of layers,
wherein the analysis circuitry calculates a relative concentration so as to satisfy a maximum smoothness condition that the relative concentration of chemical species of the sample smoothly changes in the plurality of layers of the multilayer body in minimizing the sum of square deviations.

2. The data analysis device according to claim 1, wherein the maximum smoothness condition is a condition that a sum of squares of differences in the relative concentrations between adjacent layers is minimized for all chemical species and all layers in the multilayer body.

3. The data analysis device according to claim 1, wherein the analysis circuitry applies a charge neutral condition related to the chemical species in addition to the maximum smoothness condition in minimizing the sum of square deviations.

4. The data analysis device according to claim 3, wherein the analysis circuitry optimizes a device constant that is a parameter related to the measurement device such that the sum of square deviations between a value obtained by multiplying the theoretical value of the response signal by the device constant and the measured value of the response signal is minimized.

5. The data analysis device according to claim 4, wherein the analysis circuitry alternately repeats a first arithmetic operation optimizing the relative concentration by fixing the device constant and a second arithmetic operation optimizing the device constant by fixing the relative concentration, and obtains the depth profile from the relative concentration when results of the first arithmetic operation and the second arithmetic operation converge.

6. The data analysis device according to claim 1, wherein the measurement device is an angular resolution photoelectron spectroscopic device, and the response signal is an optoelectronic signal.

7. A data analysis method comprising:
receiving, from a measurement device, a measured value of a response signal generated from a sample by incidence of a probe; and
analyzing a depth profile of the sample based on the measured value, wherein the analyzing includes
minimizing a sum of square deviations between a theoretical value of the response signal and the measured value of the response signal using the theoretical value of the response signal when the sample is modeled into a multilayer body including a plurality of layers, and
the minimizing the sum of square deviations includes calculating a relative concentration so as to satisfy a maximum smoothness condition that the relative concentration of chemical species of the sample smoothly change in the plurality of layers of the multilayer body.

8. The data analysis method according to claim 7, wherein the maximum smoothness condition is a condition that a sum of squares of differences in the relative concentrations between adjacent layers is minimized for all chemical species and all layers in the multilayer body.

9. The data analysis method according to claim 7, wherein the minimizing the sum of square deviations includes applying a charge neutral condition related to the chemical species in addition to the maximum smoothness condition in minimizing the sum of square deviations.

10. The data analysis method according to claim 9, wherein the minimizing the sum of square deviations includes optimizing a device constant that is a parameter related to a measurement device measuring the response signal such that the sum of square deviations between a value obtained by multiplying the theoretical value of the response signal by the device constant and the measured value of the response signal is minimized.

11. The data analysis method according to claim 10, wherein the minimizing the sum of square deviations includes:
repeating alternately a first arithmetic operation of optimizing the relative concentration by fixing the device constant and a second arithmetic operation of optimizing the device constant by fixing the relative concentration until results of the first arithmetic operation and the second arithmetic operation converge; and
obtaining the depth profile from the relative concentration when the results of the first arithmetic operation and the second arithmetic operation converge.

12. The data analysis method according to claim 7, wherein the measurement device is an angular resolution photoelectron spectroscopic device, and the response signal is an optoelectronic signal.

13. (canceled)

14. A recording medium in which a program is recorded, the program causing a computer to execute:
receiving, from a measurement device, a measured value of a response signal generated from a sample by incidence of a probe; and
analyzing a depth profile of the sample based on the measured value, wherein the analyzing includes
minimizing a sum of square deviations between a theoretical value of the response signal and the measured value of the response signal using the theoretical value of the response signal when the sample is modeled into a multilayer body including a plurality of layers, and
the minimizing the sum of square deviations includes calculating a relative concentration so as to satisfy a maximum smoothness condition that the relative concentration of chemical species of the sample smoothly change in the plurality of layers of the multilayer body.
